(19) United States
(12) Patent Application Publication
(10) Pub. No.: US 2019/0144937 A1
(43) Pub. Date: May 16, 2019

(54) NOVEL METHODS FOR QUANTIFYING PROTEINS USING PHAGE-BASED SEQUENCING

(71) Applicants: THE REGENTS OF THE UNIVERSITY OF CALIFORNIA, Oakland, CA (US); UNIVERSITY OF TORONTO, Toronto (CA)

(72) Inventors: James A. WELLS, San Francisco, CA (US); Samuel POLLOCK, San Francisco, CA (US); Sachdev SIDHU, Toronto (CA); Jason MOFFAT, Toronto (CA)

(73) Assignees: THE REGENTS OF THE UNIVERSITY OF CALIFORNIA, Oakland, CA (US); UNIVERSITY OF TORONTO, TORONTO, ON (CA)

(21) Appl. No.: 16/097,140

(22) PCT Filed: May 1, 2017
(86) PCT No.: PCT/US2017/030421

§ 371 Date: Oct. 26, 2018

Related U.S. Application Data
(60) Provisional application No. 62/329,730, filed on Apr. 29, 2016.

(51) Int. Cl. C12Q 1/6874; C07K 19/00

(52) U.S. Cl. CPC C12Q 1/6874; C07K 19/00; C40B 40/02; C07K 2317/565; C07K 2317/55

(57) ABSTRACT

The present invention provides methods of identifying the presence and relative abundance of a protein in or on a cell or population of cells, with the methods comprising applying to a population of cellular proteins a collection of Fab-phage particles that contain nucleic acid encoding at least one antibody Fab fragment, wherein each of the antibody Fab fragments has a known protein to which it will bind in a specific manner. After binding is allowed to occur, those Fab-phage not bound to targets are washed away and the remaining phage are propagated in bacteria before the nucleic acid within the Fab-phage is amplified and then sequenced to determine the polynucleotide sequences of the nucleic acid molecules from the Fab-phages that bound to the cellular proteins. The nucleotide sequences of the nucleic acid molecules from the Fab-phages correlate to the coding sequences of the antibody Fab fragments that are known to bind in a specific manner to a protein.

### STATEMENT REGARDING FEDERALLY SPONSORED RESEARCH OR DEVELOPMENT

This invention was made with government support under grant nos. R01 CA191018, P41 CA196276 and R01 CA154802 awarded by the National Institutes of Health. The government has certain rights in the invention.

### REFERENCE TO SEQUENCE LISTING

A computer readable text file, entitled “61818-5147-SequenceListing.txt,” created on or about 27 Apr. 2017 with a file size of about 1 kb contains the sequence listing for this application and is hereby incorporated by reference in its entirety.

### BACKGROUND OF THE INVENTION

The ability to determine cellular protein profiles has important implications for the classification of new cell types, the detection of new biomarkers, and the development of targeted therapeutics. Technologies already exist for profiling the cellular proteomes but each has drawbacks that prevent it from meeting the need for a rapid, cheap, global profiling of the cellular proteome.

Fab-phage display is usually used for selecting unique Fab-phage that bind to an immobilized target of interest. The Fab (antibody fragment) itself contains constant regions and several variable regions or complementarity-determining regions (CDRs). These CDRs can be randomized to generate a mixture of FabPhage, generally with at least 10¹⁰ different clones per mixture, where each Fab-phage possesses unique binding characteristics. By immobilizing a target, adding the Fab-phage mixture, followed by washing away any non-binding phage and propagating the binders, and perhaps repeating this process several times, a number of different Fab-phage clones can usually be found that are specific to the target of interest. This repetitive process of binding, washing, eluting, and repeating is called phage panning in the art.

Other technologies, utilize DNA sequencing instead of colony picking to identify enriched phage, but overall utilize the same repetitive “panning” scheme of binding, washing and eluting to identify specific binders.

Antibody barcoding experiments utilize DNA barcodes covalently fused to antibodies, with each barcode corresponding to the antibody to which it is attached. In this manner, antibodies bind targets on the surface of or inside of cells, the cells are washed, and amplification and sequencing is used to identify and quantify the presence of antibody targets. This method can be effective, but it is expensive to purchase large quantities of commercial antibodies, popular methods of attaching the DNA barcode leads to heterogenous antibody mixtures, and it is possible that fusing a highly negatively charged DNA molecule to the antibody could affect what that antibody can bind. Fab-phage would overcome all of these limitations by 1) serving as an inexpensive, renewable reagent and 2) containing a single DNA barcode within the phage particle that is separate from but still attached to the displayed antibody.

The present invention addresses the need for cellular proteomic profiling method that is fast, inexpensive, accessible to most labs, and can profile from hundreds to thousands of targets at once.

### BRIEF SUMMARY OF THE INVENTION

The present invention provides methods of identifying the presence and relative abundance of a protein in or on a cell or population of cells, with the methods comprising applying to a population of cellular proteins a collection of Fab-phage particles that contain nucleic acid encoding at least one antibody Fab fragment, wherein each of the antibody Fab fragments has a known protein to which it will bind in a specific manner. After binding is allowed to occur, those Fab-phage not bound to targets are washed away and the remaining phage are propagated in bacteria before the cellular proteins that specifically bind to one or more of the applied Fab-phages are isolated and separated from the bound Fab-phages and the nucleic acid within the Fab-phage is amplified and then sequenced to determine the polynucleotide sequences of the nucleic acid molecules from the Fab-phages that bound to the cellular proteins. The nucleotide sequences of the nucleic acid molecules from the Fab-phages correlate to the coding sequences of the antibody Fab fragments that are known to bind in a specific manner to a protein.

### BRIEF DESCRIPTION OF THE DRAWINGS

FIG. 1A depicts a representative Fab-phage utilized in the methods of the present invention. FIG. 1B depicts a workflow diagram of one embodiment of the methods of the present invention.

FIG. 2 provides a graph of the concept of fractional occupancy between a ligand and its binding partner.

FIG. 3 depicts the linear correlation between the number of Fab-phage and output titer observed in the “under-saturation range” (expressed as fraction output phage recovered over input phage).

FIG. 4A depicts HeLa cells that were engineered to express GFP tethered to their surface by a standard PDGF transmembrane domain. FIG. 4B depicts the Fab-phage titer in which a single anti-GFP Fab-phage was incubated with HeLaGFP cells (1M), HeLa cells (no GFP) (1M), and no cells (plastic only). The phage titer for each condition including the input is shown. The titer for phage on HeLaGFP cells was over 1,000-fold in excess over that for HeLa, which in turn was 100-fold in excess over no cells (plastic alone).

FIG. 5 depicts the results after creating, propagating, amplifying, and sequencing a defined, “mock” mixture of 32 Fab-phages designed to simulate an expected cell output.

FIG. 6 depicts the output results when subjecting the HeLaGFP cells to a library of 32 Fab-phages

FIG. 7 depicts the results of performing the methods of the present invention on MCF10A cells or MCF10A transfected with the KRAS oncogene. The results show that the fold-enrichment over background (FEOB) had a value of 19.0 for MCF10A-EV cells and 84.6 for MCF10A-KRAS cells, or a ^{˜}4.45-fold increase, of the CDCP1 gene in KRAS cells over empty vector cells. These results compare quite favorably with data using flow cytometry in which it has been reported or observed that MCF10A cells show an increase of ^{˜}4-5 times of surface CDCP1 gene upon transformation with KRAS, relative to empty vector. Moreover, a number of proteins previously undetected by other methods were observed to be upregulated in the transfected cells. For example EGFR, AXL, FGFR2 and PDGFRA are all upregulated upon KRAS transformation.

FIG. 8 depicts a workflow of the methods of the present invention being used for intracellular proteins.

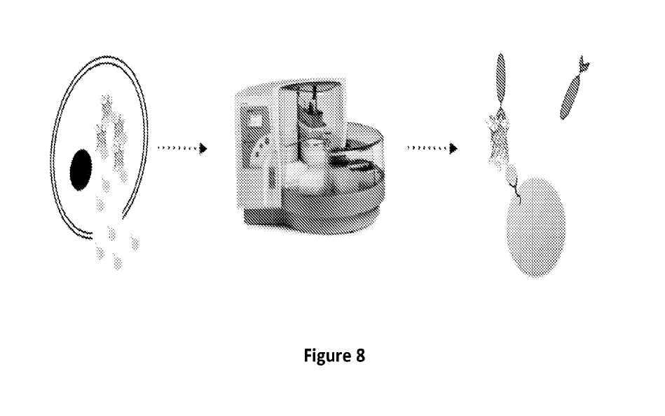

FIG. 9 depicts results from performing the methods of the present invention on HEK293T cells expressing intracellular GFP and non-expressing HEK293T. In the figure, “Control+” is GFP with a biotinylated Avitag, which was 100% biotinylated and was not treated with NHS-biotin; “Control−” is PBS alone, “NHS-biotin+GFP” is His-tagged GFP (no biotin) that was mixed with NHS-biotin to randomly biotinylate its surface lysines; “GFP lysate” is HEK293T cells expressing cytosolic GFP that were lysed and the entire lysate was biotinylated using NHS-biotin; “Control lysate” is HEK293T cells not expressing cytosolic GFP that were lysed and the entire lysate was biotinylated using NHS-biotin; “Biotin-GFP+lysate control” is identical to control lysate, except that similar amount of biotinylated GFP as the control+condition was added. It was observed that a small but significant increase in titer between cells that express GFP over those that do not, demonstrating the feasibility of this embodiment of the technique.

FIG. 10 depicts PhaNGS profiling showing differences in the cell surfaceomes at diagnosis and relapse in a patient with ALL. (A) Samples were obtained from a patient at diagnosis with ALL, labeled LAX7. After chemotherapy treatment the patient relapsed and samples were obtained, labeled LAX7R. (B) PhaNGS profile for 192 different Fab-Phage directed to 58 different surface protein targets binding to LAX7 (blue) or LAX7R (red). Regions showing the largest changes in Fab-phage binding, either increased or decreased, between the two patient cells are presented in magnification call outs. Experiments were conducted in quadruplicate, normalized to background, and corrected for variable input quantities (as performed in previously described PhaNGS experiments). (C) The same data as in (B) showing the signal ratio for LAX7R to LAX7 is displayed as a fold-change chart where the most up-regulated targets are displayed on the left and most down-regulated are displayed on the right.

FIG. 11 depicts measuring proteomic changes by PhaNGS in the Myc repressible cell line, P493-6, where addition of tetracycline can turn off Myc expression. (A) Experimental scheme for the P493-6 cell line in Myc-ON, OFF, or BACKON conditions. After harvesting cells from the ON state, Myc was knocked down for 48 hrs with the addition of tetracycline (100 ng/μL), twice per day. The OFF state was harvested, the tetracycline was washed out, and the cells were allowed to recover for six days before the BACKON condition was harvested. (B) The extended bar chart shows the results of the PhaNGS profiling for the ON-OFF-BACKON experiments, shown by blue, red, and green bars, respectively. Regions of interest are presented in magnification call outs. Each of these four targets has three unique Fabs for each target. Experiments were conducted in quadruplicate and background corrected.

FIG. 12 depicts single-cell PhaNGS with P493-6 cells (in the ON state). (A) Flow cytometry histograms for ROR1 (left panel) and insulin receptor (INSR) (right panel) on P493-6 cells. ROR1 showed a major high expression peak and a minor low expression peak, but INSR showed one narrow high expression peak. Flow data from two replicate experiments run several months apart are shown for ROR1 to demonstrate the reliable observation of the minor peak. (B) Results from single-cell PhaNGS using ROR1 and INSR Fab-phage on 84 individual P493-6 cells. Similar to the flow cytometry data, the INSR signal is uniformly high and mono disperse, whereas there are two expression populations for ROR1 and bimodal with the majority of cells (^{˜}75%) express high levels, and a minority (^{˜}25%) express low levels.

FIG. 13 depicts an experimental design for using the methods for in vivo identification of proteins.

### DETAILED DESCRIPTION OF THE INVENTION

The present invention, referred to as phage-based next-generation sequencing (“PhaNGS”), is a technique used to compare the abundance of one or more cellular proteins between two or more cell populations of interest, or of a single cell population of interest or of a single cell of interest. The central component of the present invention is the Fab-phage, which is a bacteriophage engineered to display antibody fragments from their pill coat protein.

The methods of the present invention are used to detect and, in some embodiments, quantify a multiplicity of proteins from a population of cells. In certain embodiments, the methods comprise at least 10 different Fab-phages, with each Fab-phage corresponding to a different protein to which the Fab-phage binds. In certain embodiments, the methods comprise at least 20 different Fab-phages, with each Fab-phage corresponding to a different protein to which the Fab-phage binds. In certain embodiments, the methods comprise at least 30 different Fab-phages, with each Fab-phage corresponding to a different protein to which the Fab-phage binds. In certain embodiments, the methods comprise at least 40 different Fab-phages, with each Fab-phage corresponding to a different protein to which the Fab-phage binds. In certain embodiments, the methods comprise at least 50 different Fab-phages, with each Fab-phage corresponding to a different protein to which the Fab-phage binds. In certain embodiments, the methods comprise at least 60 different Fab-phages, with each Fab-phage corresponding to a different protein to which the Fab-phage binds. In certain embodiments, the methods comprise at least 70 different Fab-phages, with each Fab-phage corresponding to a different protein to which the Fab-phage binds. In certain embodiments, the methods comprise at least 80 different Fab-phages, with each Fab-phage corresponding to a different protein to which the Fab-phage binds. In certain embodiments, the methods comprise at least 90 different Fab-phages, with each Fab-phage corresponding to a different protein to which the Fab-phage binds. In certain embodiments, the methods comprise at least 100 different Fab-phages, with each Fab-phage corresponding to a different protein to which the Fab-phage binds. In certain embodiments, the methods comprise at least 150 different Fab-phages, with each Fab-phage corresponding to a different protein to which the Fab-phage binds. In certain embodiments, the methods comprise at least 200 different Fab-phages, with each Fab-phage corresponding to a different protein to which the Fab-phage binds. In certain embodiments, the methods comprise at least 250 different Fab-phages, with each Fab-phage corresponding to a different protein to which the Fab-phage binds. In certain embodiments, the methods comprise at least 300 different Fab-phages, with each Fab-phage corresponding to a different protein to which the Fab-phage binds. In certain embodiments, the methods comprise at least 350 different Fab-phages, with each Fab-phage corresponding to a different protein to which the Fab-phage binds. In certain embodiments, the methods comprise at least 400 different Fab-phages, with each Fab-phage corresponding to a different protein to which the Fab-phage binds.

The methods of the present invention rely on the use of set or collection of pre-defined Fab-phages to determine the presence or absence of one or more target proteins. The collection of Fab-phages used herein is thus not used to determine or discover new binding agents that can bind to target molecules. Rather, the Fab-phage system described and used herein is comprised of binding entities that are known to bind to a target, i.e., the binding portions of the Fab-phage molecules of the collection of Fab-phages used and described herein are “predefined.”

Accordingly, the systems and methods described herein are not phage display libraries. As is well understood in the art, a phage display system comprises bacteriophages on which potential binding entities are “displayed” on the phage coat. It is unknown, however, if the binding entities displayed on the phages are capable of binding a pre-selected target. Thus, a typical phage display is used to screen for potential ligands to a known target. In contrast, the system and methods of the present invention utilize Fab-phages, as defined herein, wherein the binding portion of the Fab-phages are already known to bind to a specific protein. Thus, the present invention provides a screening system that has a completely different configuration than that of a phage display system.

As used herein, the term “Fab” when used to in connection with the term Fab-phage, is used to mean a fragment of an antibody that contains at least a variable heavy and light chain arm of an antibody that is responsible for an antibody's ability to bind to an antigen. The term “Fab,” when used in connection with Fab-phage can therefore mean an scFv fragment, it can mean a “Fab fragment” of a full length antibody, or it can mean an “affinity reagent.” A single chain Fv fragment (scFv) is a well understood term of art and means a single chain polypeptide that contains an antibody variable heavy chain and an antibody variable light chain that are linked to one another with a linker peptide. A Fab fragment is also a well-understood term of art and means a fragment of an antibody that contains the variable heavy and variable light chains, as well as one heavy chain constant region and the light chain constant region. Fab fragments are generally composed of two separate polypeptide chains that are coupled to one another through a disulfide cysteine bind between the two chains. In one embodiment, the Fab-phage comprises an scFv that corresponds to the binding region of a full length antibody. As used herein, the term affinity reagent is used to mean an antibody-like protein that can be displayed on the surface of the phage. In another embodiment, the Fab-phage comprises a Fab fragment that corresponds to the binding region of a full length antibody. The present invention utilizes a collection of a multiplicity of Fab fragments that are displayed on the surface coat of a viral particle through the use of a phagemids. A phagemid typically encodes for a single coat protein, called pill, and the present invention fuses a coding region of a Fab fragment to the pill protein. The construct encoding the Fab-pill fusion may or may not include a region encoding a small, flexible linker of amino acids separating the two.

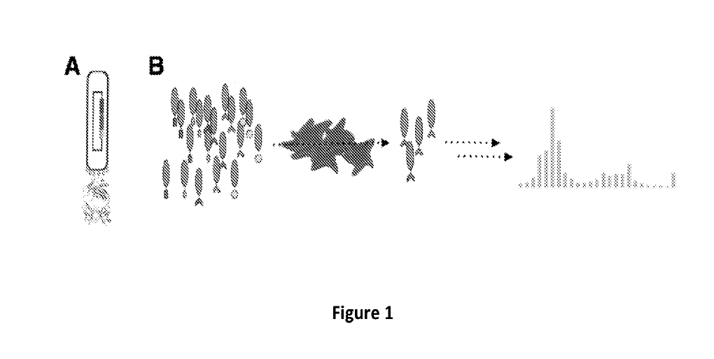

The phagemid DNA encoding the pill-Fab fusion is transfected into bacteria using common, routine techniques for introducing nucleic acids into bacteria. The phagemids used herein may or may not include other components of a viral genome. A “helper phage,” such as but not limited to VCSM13 or M13K07, is then utilized to infect the host and enable virus production, including viral packaging of the phagemid into a virus particle that displays the Fab fragment on its surface coat. Accordingly, the term “Fab-phage,” as used herein, means a virus particle that displays at least one Fab fragment on its surface and includes or encapsulates the phagemid or circular plasmid DNA (FIG. 1A). The surfaces of cells are large enough to accommodate the binding of many thousands of Fab-phage, which are generally less than 10 nm wide and hundreds of nm long.

In one embodiment, the “Fab” portion of the Fab-phage is an scFv

Fab-phages that bind to specific extracellular targets, for example, from a “curated library” and Fab-phage negative controls, i.e., Fab-phages that do not bind to anything present on the surface of cells, such as but not limited to anti-transcription factor Fab-phage, are then mixed with a population of cells (FIG. 1B). If the target of the Fab-phage is present in or on the cells, that specific Fab-phage is retained while unbound Fab-phage are washed away.

The washing step generally include re-suspending cells in a buffer, such as but not limited to PBS, centrifuging cells into a pellet, disposing of the supernatant, and repeating. In one embodiment, tubes, plates, or other vessels into which the cells are transferred are composed of a plastic or other material to which a phage will typically bind non-specifically. In one embodiment, when the techniques are to be applied to a single cell, single cells to which Fab-phages are bound are sorted into single wells. In select embodiments, the washing steps described above are performed more than once.

Once isolated, the Fab-phages bound to cells are released from the cells using any technique designed to interfere with or disrupt phage-cell binding. In one embodiment, the cells are treated with acid to release the Fab-phage from the cell to which it is bound. The released phages can then be propagated, for example in bacteria, or the DNA from the phages is amplified directly from the surface of the cell or bead to which the Fab-phage is attached. The direct amplification of the phage DNA or the “indirect amplification” of the DNA though bacterial propagation is intended to increase the amount of DNA for subsequent sequencing. In one embodiment, the Fab-phage DNA is amplified directly. In another embodiment, the Fab-phage DNA is amplified through bacterial propagation.

In direct amplification of the Fab-phage DNA, the phage's hypervariable complementarity determining heavy-chain 3 region (CDR H3 region) is amplified using routine amplification techniques, such as but not limited to PCR, which utilize custom primers over a certain number of amplification cycles. In select embodiments, the number amplification cycles of the isolated Fab-phage DNA is at least 1, 2, 3, 4, 5, 6, 7, 8, 9, 10, 11, 12, 13, 14, 15, 16, 17, 18, 19, 20, 21, 22, 23, 24 25, 26, 27, 28, 29, 30 or even more cycles. In select embodiments, the number of amplification cycles is few enough to not introduce a statistical bias during the quantification of the amplicons. As used herein, an “amplicon” is used as it is in the art and means a nucleic acid, such as an RNA or DNA, that is amplified during an amplification reaction.

Once generated the amplicons can then be sequenced to identify which Fab-phage DNA sequences were enriched during the isolation and amplification processes, relative to the negative controls. The sequence identity of the isolated Fab-phage will then permit detection and identification of specific proteins present on or in the cell or cells of interest. Any technique for sequencing DNA can be used in the methods of the present invention. In one embodiment, the sequencing techniques comprise any one or more of the “next generation sequencing” (NGS) techniques. NGS techniques are high-throughput DNA sequencing techniques, such as but not limited to, Illumina sequencing, Roche 454 sequencing, Proton/PGM sequencing and SOLiD sequencing.

In select embodiments, the present methods comprise the use of custom primers. In general, the structures of the primers of the present invention comprise one or more indexing sequences and at least one complementary sequence which allows the primer to bind to the phagemid. In one embodiment, the polynucleotide sequence of one of the indexing nucleic acids of the present invention comprises or consists of the polynucleotide sequence of SEQ ID NO:1: AATGATACGGCGACCACCGAGATCTACACNNNNNNNNNNNNTGAGGACACTGCCGTCTATTATTGTGCTCG C (SEQ ID NO:1). In another embodiment, the polynucleotide sequence of one of the indexing nucleic acids of the present invention comprises or consists of the polynucleotide sequence of SEQ ID NO:2:

(SEQ ID NO: 2)
GACTACTGGGGTCAAGGAACCCTGGTCAAGATCGGAAGAGCACACGT
CTGAACTCCAGTCACNNNNNNNNNNNNATCTCGTATGCCGTCTTCTG
CTTG.

In yet another embodiment, the primers comprise a polynucleotide sequence comprising SEQ ID NOs: 1 and 2. In one specific embodiment, the primers flank one or more “intervening polynucleotide sequences” that are inserted in between SEQ ID NO:1 and SEQ ID NO:2 in the resulting amplicon. In one embodiment, SEQ ID NO:1 is 5′ to the intervening sequence and SEQ ID NO:2 is 3′ to the intervening sequence. In another embodiment, SEQ ID NO:2 is 5′ to the intervening sequence and SEQ ID NO:1 is 3′ to the intervening sequence.

An intervening sequence can be any sequence that is the target for amplification. In select embodiments, the coding region of an H3 variable region of a Fab complementarity determining region (CDR) is the intervening sequence. As is well-known in the art, there are myriad potential scaffold sequences which flank the intervening sequence. Thus, the primers of the present invention can comprise virtually any sequence which serves to complement the flanking scaffold sequences, provided that the at least one of the nucleic acids of SEQ ID NO:1 and/or SEQ ID NO:2 are included in the primer.

The intervening sequence of the primer need not be directly adjacent (5′ and/or 3′) to either of the nucleic acids of SEQ ID NO:1 or 2. For example, the primers may or may not include “filler” nucleotides between the intervening nucleic acid and one of the indexing primers. In select embodiments, the primers of the present invention contain 0, 1, 2, 3, 4, 5, 6, 7, 8, 9, 10, 11, 12, 13, 14, 15, 16, 17, 18, 19, 20, 20, 40, 50, 60, 70, 80, 90, 100 of hundreds or more filler nucleotides in between the intervening sequence and at least one of the indexing primers.

The design of the primers provided in the present invention allows for “indexing” of the Fab-phage DNA isolated from the Fab-phages used in the methods of the present invention. In one embodiment, the Fab-phage DNA is indexed at least once during amplification, identification and/or quantification. In another embodiment, the Fab-phage DNA is indexed at least twice during amplification, identification and/or quantification. In another embodiment, the Fab-phage DNA is indexed at least three times during amplification, identification and/or quantification.

As used herein, indexing is a method of collecting or clustering sequencing outputs into the same experiment or set of experiments by providing a unique nucleotide sequence for each experimental output. For instance, if one were to perform the methods of the present invention on e.g., negative control cells, cells given treatment 1, and cells given treatment 2, the Fab-phage DNA could be indexed three different ways such that all experiments (negative, treatment 1, and treatment 2) could be sequenced together in the same “run” yet allow the data from each experiment or experimental condition to be isolated. The sequencing output for the negative control would provide a unique nucleotide sequence such that negative control is associated, or indexed, to a unique sequence for the control. The sequencing output for treatment 1 would provide a unique nucleotide sequence such that treatment 1 is associated, or indexed, to unique sequence 1. Similarly, the sequencing output for treatment 2 would provide a unique nucleotide sequence such that treatment 2 is associated, or indexed, to unique sequence 2.

In select embodiments of the present invention, the Fab-phage DNA is indexed twice, “dual indexed” such that each experiment receives two, separately sequenced indices. In another embodiment of the present invention, the Fab-phage DNA is indexed once, “single indexing” such that each experiment receives only one separately sequenced index. Dual indexing allows for more durable separation of data provided in the sequencing experiments for each experimental index. That is, requiring the observation of two indices before assigning a given read to an experiment reduces the chance of mis-assignment compared to requiring observation of only a single index. Thus, if more accurate quantification of the bound Fab-phage DNA is desired, sequencing methods utilizing dual indexing would be recommended.

Accordingly, quantification of the cellular proteins is also possible using the methods of the present invention. In one embodiment, the methods comprise quantifying the number of cellular proteins in or on a given cell or population of cells. As used herein, the quantification may be relative or absolute and may be expressed as number, percentage, ratio and the like. The quantity may simply be the measured levels of DNA without any additional measurements or manipulations. Alternatively, the quantities may be manipulated mathematically or in an algorithm, with the algorithm designed to correlate the measured DNA value to the quantity of cellular proteins in the cell or population of cells or on a per cell basis. The quantity may be expressed as a difference, percentage or ratio of the measured value of the cellular protein compared to another protein including, but not limited to, a negative or positive control.

The methods of the present invention can also be used to measure or monitor the presence of one or more specific cell protein over time. For example, the methods of the present invention can be used to detect and/or quantify the presence of a cellular protein at various time points by performing the methods at more than one time point. For example, a cell or population of cells can be harvested from a subject at more than one time point, and the methods of the present invention can be applied to each cell or population of cells to determine if a specific cellular protein is changing over time or in response to the application or withdrawal of a treatment or stimulus to the cells. Such information could then be used to formulate an individualized treatment for a given subject.

Thus, the present invention also includes methods of monitoring the presence of a cellular protein in a cell or population of cells, with the methods comprising determining the identity and/or quantity of one or more cellular proteins more than once over a period of time. The cells can be isolated from a subject at various points in time and subjected to the methods of the present invention at more than one time point and to determine if the levels of a specific cellular protein in or on the cells is increasing or decreasing over time. In some embodiments the monitoring and diagnostic methods of the present invention will comprise determining the identity and/or quantity of specific cellular proteins two, three, four, five, six, seven, eight, nine, 10 or even more times over a period of time, such as a week or more, two weeks or more, three weeks or more, a month or more, two months or more, three months or more, four months or more, five months or more, six months or more, seven months or more, eight months or more, nine months or more, 10 months or more, 11 months or more, a year or more, two years or more, three years or more, four years or more, five years or more, six years or more, seven years or more, eight years or more, nine years or more or even 10 years or longer.

In one embodiment, the methods of the present invention can be used to determine an increase or decrease in one or more cellular proteins over time or in response to a specific condition or in response to a treatment of a specific condition. For example, the methods can be used to assess levels of cellular proteins, e.g., cell surface proteins, in a subject that is receiving treatment for an abnormal condition such as cancer. The abnormal condition or treatment thereof is not critical to the invention in that the invention is deigned to assess changes in cellular proteins over time, including the determination of there being no change, regardless of the cause of the change in cellular proteins. In another example, the methods can be used to assess levels of cellular proteins in response to a genetic change in a population of cells, such as the expression or repression of a specific gene, e.g., myc, or to an epigenetic change that may affect gene expression in the cell population. Similarly, the genetic change is not critical to the invention in that the invention is deigned to assess changes in cellular proteins over time, including the determination of there being no change, regardless of the cause of the change in cellular proteins.

As used here, the subject from which the cells are taken can be a human or non-human animal. If the cells are non-human, the Fab-phages may still be human Fabs, thus the methods of the present invention can be used to assess cross-reactivity of human antibodies (or antibody fragments) with non-human, e.g., mouse, antigens. In other words, if a Fab-phage of the present invention, wherein the Fab is of human origin, binds to a cellular protein on, e.g., a mouse cell, and is then quantified according to the methods of the present, this binding would be an indication that the human Fab, and therefore the “parent antibody,” is cross-reactive to mice. Accordingly, the methods herein could be used to determine cross-reactivity of antibodies amongst two or more separate species.

In one embodiment, the subject's expression levels of one or more of a specific cellular protein can be compared to the expression levels of the same protein that are deemed to be “normal” levels. To establish the levels of a normal individual, an individual or group of individuals may be first assessed for symptoms or signs of a specific condition, e.g., diabetes, to establish that the individual or group of individuals has normal, healthy or acceptable levels of a specific protein is associated with a specific condition or disease. Once established, the expression levels of the protein of interest for an individual or group of individuals can then be determined to establish “normal levels.” In one embodiment, normal expression levels can be ascertained from the same subject when the subject is deemed to be normal or healthy with no detectable signs of (clinical or otherwise) of the specific condition or disease in question. In one embodiment, “normal levels” are assessed in the same subject from whom the sample is taken prior to the onset of any measureable, perceivable or diagnosed condition. That is, the term “normal levels” with respect to expression levels of a specific cellular protein can be used to mean the subject's baseline levels prior to the onset of any condition. The expression levels of the specific cellular protein in question can then be reassessed periodically and compared to the subject's baseline levels.

The interaction between a ligand and its binding partner in biology can be expressed as an equilibrium. Ligands will associate with binding partners, leaving one with free ligand (A), free binding partner (B), and ligand-binding partner complex (AB) in any given system as shown in Equation 1.

AₓB_{y} [symbol] xA+yB Equation 1

This relationship can be elaborated to contain affinity information by describing the dissociation constant (Kd) as shown in Equation 2.

$${K}_{d} = \frac{{{[ A ]}^{x} [ B ]}^{y}}{[ {A}_{x} {B}_{y} ]} \qquad Equation 2$$

The dissociation constant is a measure of the binding affinity of the reaction, and is directly defined as the concentration of ligand at which half of the binding partner is bound by ligand. Note that the concentration of ligand and binding partner may change, but the dissociation constant, or Kd, does not for a given interaction.

With respect to the methods of the present invention, one value that can be useful in quantifying Fab-phage DNA, and therefore the number of binding partners that bind to a specific Fab-phage and to which a Fab-phage binds, is the “fractional occupancy” of binding partner to its ligand. The equation for expressing ligand fractional occupancy can be expressed as Equation 3, below:

$$x = \frac{- ( y + z + Kd ) + \sqrt{{( y + z + Kd )}^{2} - 4 yz}}{- 2} \qquad Equation 3$$

Where y is [M total], z is [L total], and x is [ML], where “M” standards for macromolecule or binding partner, “L” stands for ligand (such as a Fab-phage), and ML is the complex of the two. Under certain conditions, such as when the ligand or binding partner is in over 10-fold excess over the other and if the species (binding partner or ligand) in excess is either 10-fold above or below the Kd of the interaction, then Equation 3 may be simplified to Equation 4 to provide a fractional occupancy:

$$Fractional occupancy = \frac{[ L ]}{[ L ] + Kd} \qquad Equation 4$$

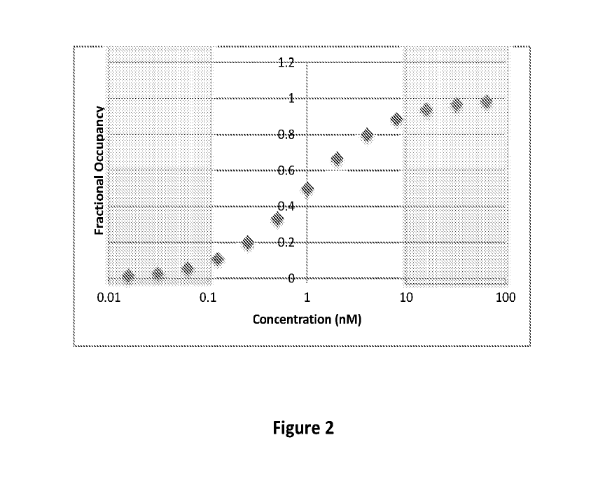

FIG. 2 provides Equation 4 in a graphical form when the Kd is 1 nM. In FIG. 2, the shaded regions lie 10-fold above or below the Kd in concentration (over-saturation and under-saturation regimes respectively).

Standard technologies that monitor protein abundance (quantity), including those that are quantitative, such flow cytometry, usually utilize the over-saturation regime in which there is far more ligand, e.g., antibody, Fab fragment, etc., than binding partner. The binding phase of standard quantitative methods is performed without consideration of fractional occupancy because any concentration approximately 10-fold above the Kd of the interaction is assumed to produce the same result. It is not believed that fractional occupancy is considered in any other phage display techniques because phages have been used only to identify Fabs that bind to particular targets.

Table 1 provides fractional occupancy of a given a concentration of ligand assuming a Kd of 1 nM between ligand and binding partner.

TABLE 1

| | Fractional | |
|---|---|---|
| [L] (nM) | Occupancy | Fold change from above |
| 16 | 0.94 | |
| 8 | 0.89 | 1.06 |
| 4 | 0.80 | 1.11 |
| 2 | 0.67 | 1.20 |
| 1.00 | 0.50 | 1.33 |
| 0.50 | 0.33 | 1.50 |
| 0.25 | 0.20 | 1.67 |
| 0.13 | 0.11 | 1.80 |
| 0.06 | 0.06 | 1.89 |
| 0.03 | 0.03 | 1.94 |
| 0.016 | 0.02 | 1.97 |

Based on the concept of fractional occupancy, one aspect of the present invention provides for performing the methods of the present invention in binding conditions that are less than fully saturated, i.e., in “under saturation conditions.” As used herein, the term “under saturation regime” is used to indicate that the ligand or binding partner is in excess over the other and when the species (binding partner or ligand) in excess is also above or below the Kd of the interaction. In a specific embodiment, an “under saturation regime” or “under saturated range” is when ligand or binding partner is in an over 10-fold excess over the other and when the limiting species (binding partner or ligand) is 10-fold below the Kd of the interaction. For example, using the hypothetical values in Table 1 a concentration below 0.1 nM Fab-phage (L) would be considered in the “under saturated regime” or “under saturated range.” Because the concentration of the Fab-phage is so low, it is possible to utilize, for example, at least 10 different Fab-phage clones against a single binding partner (M) and still not exceed the maximum occupancy of the binding partner (M). Moreover, utilizing the methods of the present invention in an under-saturation regime provides for the ability to quantify the cellular protein (ligand) abundance in or on cells.

Another aspect of the present invention provides for performing the methods of the present invention in binding conditions that are more than fully saturated, i.e., in “over saturation conditions.” As used herein, the term “over saturation regime” is used to indicate that the ligand or binding partner is in excess over the other and when the species (binding partner or ligand) in excess is also above the Kd of the interaction. In a specific embodiment, an “over saturation regime” or “over saturated range” is when ligand or binding partner is in an over 10-fold excess over the other and when the limiting species (binding partner or ligand) is 10-fold above the Kd of the interaction.

The quantitative data derived from the methods described herein may or may not be, normalized and/or corrected. For example, the quantities developed herein may be corrected using the relative or absolute binding affinities of each Fab-phage to its specific binding partners. For example, the observed or calculated quantities could be adjusted upwards if the binding affinity for a particular Fab-phage is weaker than another Fab-phage. Similarly, the observed or calculated quantities could be adjusted downwards if the binding affinity for a particular Fab-phage is stronger than another Fab-phage.

In additional embodiments, the methods of the present invention can be used to determine the presence of one or more intracellular proteins and “free proteins,” such as proteins found in the circulation, cerebrospinal fluid, extracellular tissue, etc. For example, the proteins can be exposed to the Fab-phages of the present invention by, for example, lysing the cells, if intracellular proteins are the target, and immobilizing the intracellular proteins onto solid surfaces, such as, but not limited to magnetized or non-magnetized beads, cell culture surfaces and the like. Of course, lysing cells would not be necessary if free proteins are the target. Any technique used to attach protein to surfaces can be used, including but not limited to biotinylation followed by streptavidin binding.

In some embodiments, cells are lysed and the proteome is biotinylated such that the proteome can be captured using, for example magnetic streptavidin beads. In the alternative, the free proteins in the sample are biotinylated such that the proteins within the sample can be captured with beads. The beads can then be subjected to the methods of the present invention to identify and/or quantify intracellular proteins that bind to specific Fb-phages.

In another embodiment, the methods include a “pre-enrichment step” using phage precipitation methods. Such a pre-enrichment step could be used to simplify the proteome loaded onto the beads, thus reducing the signal to noise ratio of any analytics. In one specific embodiment, the cellular lysates are biotinylated and the Fab-phages are mixed with the lysate, which would lead to formation of complexes between phage and biotinylated target. The phage would then be precipitated using standard PEG/NaCl methods, and the phage-biotinylated protein complexes could then be loaded onto streptavidin beads. After loading, the beads would be washed and the phages would be eluted and titered.

In additional embodiments, the methods of identifying and/or quantifying an intracellular protein may further include a pre-fractionation step whereby, for example, the nucleus is separated from the rest of the cell and intranuclear proteins could be identified and/or quantified using the methods of the present invention.

The present invention also provides for methods of identifying one or more target molecules in a mixture, with the methods comprising contacting a sample with a collection of binding protein DNAs (BPDNAs), removing non-binding BPDNAs, for example by washing, and identifying the BPDNAs bound to the target. As used herein a binding protein DNA (BPDNA) is a genetically encoded binding polypeptide (BP) that is known to bind to a specific molecule and is non-covalently linked to its respective coding gene (DNA). As used herein, binding proteins (BPs) can include any polypeptide that contains one or more domains that bind to a target. One example of a BP is an antibody or antibody fragment.

In select embodiments, the binding protein or peptide is genetically encoded and displayed from a virus or a cell. In additional embodiments, the BPDNA is derived from phage display, yeast display, mammalian cell display or bacterial display.

Once BPDNA is bound to the target, its identity can be obtained by analyzing the DNA to which the BP is linked, such as but not limited to NGS techniques described herein. Using techniques described herein, the targets can also be quantified.

In select embodiments, the target is a biomolecule, a peptide, a protein, a small molecule, a carbohydrate or a lipid. If the target molecule is a protein, the protein can be a soluble protein, an intracellular protein, an extracellular protein, a plasma-derived protein or a cell surface protein. Examples of proteins that can be target molecules include proteins having randomized sections within a constant scaffolding, such as but not limited to fibronectin III domains, Darpins, Protein A, Protein G and ubiquitin.

In additional embodiments, the sample containing the target molecule can be an artificial support onto which the target molecule is attached.

The present invention also provides for methods for identifying one or more target molecules in a mixture comprising contacting a sample with a collection of genetically encoded binding polypeptides that are known to bind to specific molecules and are covalently linked to their respective coding gene, removing non-binding polypeptides, and identifying the polypeptide/coding gene complex bound to the target. In specific embodiments, the polypeptide/coding gene complex is a plasmid display or ribosome display.

### EXAMPLES

### Example 1

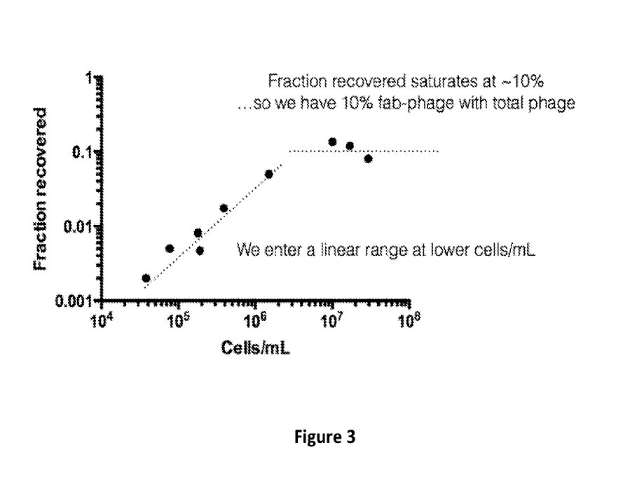

HeLa cells over-expressing surface GFP (HeLaGFP, between 10⁵ and 10⁶ receptors per cell) were mixed with ^{˜}2×10⁹ anti-GFP Fab-phage/mL, wherein the GFP/anti-GFP has a Kd of ^{˜}1 nM. Results are shown in FIG. 3.

In this HeLaGFP experiment, at 10⁶ cells and below, there is a linear correlation between number of phage and output titer observed in the “under-saturation range” (expressed as fraction output phage recovered over input phage). As the number of cells is increased, the graph begins to level off, indicating that reaction is moving towards the saturated range.

### Example 2

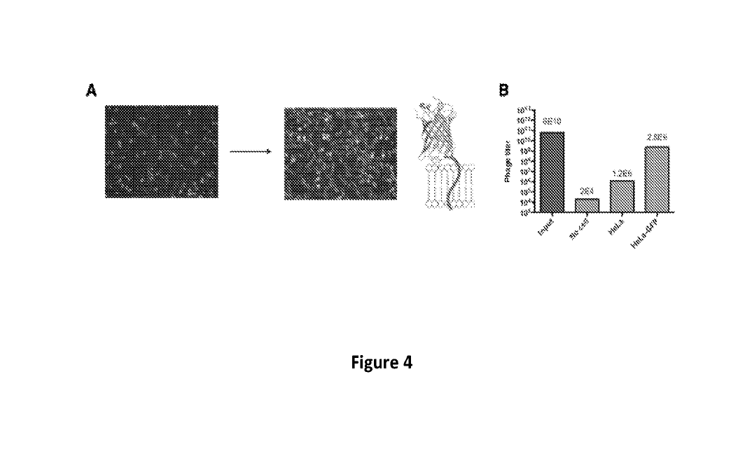

HeLaGFP cells HeLa cells that were engineered to express GFP tethered to their surface by a standard PDGF transmembrane domain (FIG. 4A). The HeLaGFP cells (1M), HeLa cells (no GFP) (1M), and no cells (plastic only) were incubated with a stock of anti-GFP Fab-phage. The results are shown in FIG. 4A and FIG. 4B.

In FIG. 4B, the phage titer for each condition including the input is shown. The titer for phage on HeLaGFP cells was over 1,000-fold in excess over that for HeLa, which in turn was 100-fold in excess over no cells (plastic alone).

### Example 3

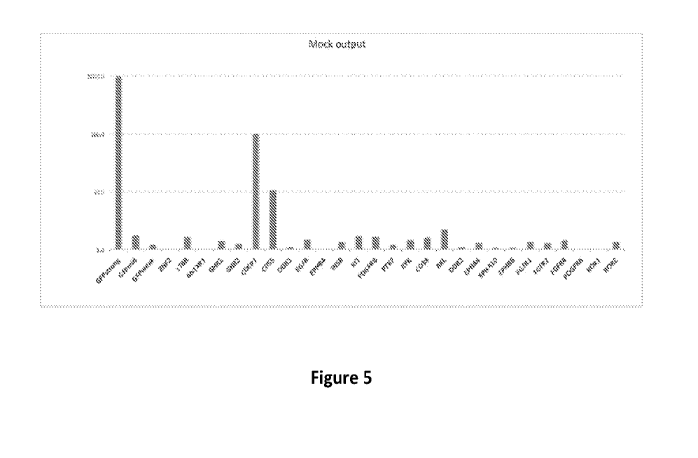

A “mock output” of a defined NGS mixture was generated. This mock output comprised a mixture of 32 Fab-phages. The mixture was made up of each Fab-phage clone at approximately 2×10⁹ cfu/mL and was diluted to 2×10⁵ cfu/mL. The following phage clones were then added back in one at-a-time: GFPstrong at 2×10⁸ cfu/mL, CDCP1 at 2×10⁷ cfu/mL and CD55 at 2×10⁶ cfu/mL. This mixture, along with the 2×10⁹ cfu/mL of G32 input from which it was derived, was then amplified using 18-cycles of PCR or propagated for its Phase I amplification. The mixture was then subjected to a 12-cycle amplification using the custom primers described above and was submitted for NGS. The results are shown in FIG. 5, and in Table 2 below:

TABLE 2

| | | | | Raw mock |
|---|---|---|---|---|
| | Raw input | Raw mock | Raw input | output |
| | counts | output counts | counts | counts |
| Clone name | (propagation) | (propagation) | (PCR) | (PCR) |
| ZNF2 | 1691824 | 15269 | 2543900 | 25489 |
| GFPstrong | 511704 | 4609999 | 1250209 | 4705648 |
| CDCP1 | 1164028 | 1060644 | 1563816 | 807062 |
| CD55 | 1140005 | 110958 | 1806090 | 113910 |

Counts for each clone were divided by (normalized to) the ZNF2 negative control to produce the data in Table 3.

TABLE 3

| | Normalized | Normalized | | Normalized |
|---|---|---|---|---|
| | input | mock | Normalized | mock output |
| Clone | counts | output counts | input counts | counts |
| name | (propagation) | (propagation) | (PCR) | (PCR) |
| ZNF2 | 1.0 | 1.0 | 1.0 | 1.0 |
| GFPstrong | 0.3 | 301.9 | 0.5 | 184.6 |
| CDCP1 | 0.7 | 69.5 | 0.6 | 31.7 |
| CD55 | 0.7 | 7.3 | 0.7 | 4.5 |

Next, values were corrected by accounting for each of the clones differing in their initial abundance at the beginning of the experiment. That is, when the phages are propagated individually for use in these experiments, their concentrations are close but not identical. To correct for initial abundance, the normalized output value were divided by their normalized input values (see column 2 in Table 3). Once the values are corrected for initial abundance, the following data is shown in Table 4:

TABLE 4

| | Normalized and corrected | Normalized and corrected |
|---|---|---|
| | mock output | mock output values |
| | values (propagation) | (PCR) (aka |
| | (aka fold-enrichment | fold-enrichment over |
| Clone name | over background) | background) |
| ZNF2 | 1.0 | 1.0 |
| GFPstrong | 998.2 | 375.7 |
| CDCP1 | 101.0 | 51.5 |
| CD55 | 10.8 | 6.3 |

### Example 4

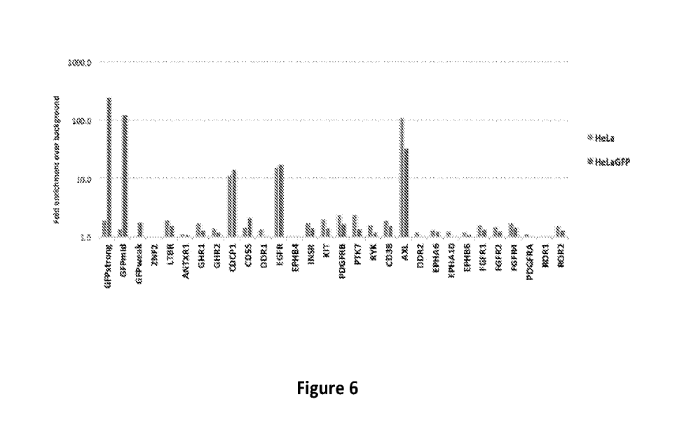

A full assessment of cell surface proteins test was assessed according to the methods of the present invention and performed on HeLa cells and HeLaGFP cells as described abvove. The fold-enrichment over background for each clone for HeLa and HeLaGFP is shown in FIG. 6.

Put another way, with the affinity corrections the three GFP bars would be at an equivalent height (due to the fact that there's one and only one concentration of surface GFP on the cells investigated).

### Example 5

MCF10A cells transformed with the oncogenic KRAS (MCF10A-KRAS) typically display higher levels of the protein CDCP1 on their surface, relative to their untransformed counterparts. To test the methods described herein, MCF10A cells with and without the KRAS oncogene being transfected were subjected to the methods of the present invention.

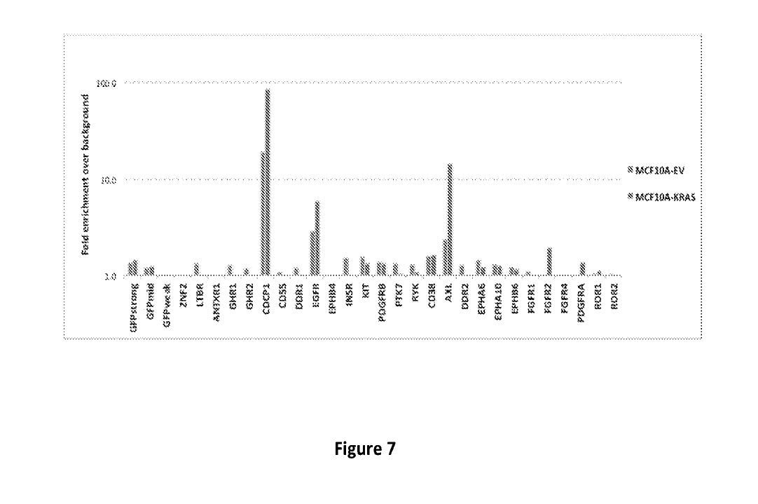

Specifically, MCF10A or MCF10A-KRAS cells were mixed with a collection of 32 different Fab-phages against surface proteins. After allowing the Fab-phages to bind, the cells were washed and the phages were eluted from the bound cells. After propagating the eluted phage using bacteria, the DNA within the Fab-phage, in particular, the H3 region was sequenced via an Illumina HiSEQ insturment. The results are shown in FIG. 7.

It had been previously observed using flow cytometry that the abundance of CDCP1 in MCF10A-KRAS cells increases 4-5-fold on MCF10A cells upon transformation with KRAS, relative to empty vector. In this experiment, the fold-enrichment over background (FEOB) had a value of 19.0 for MCF10A-EV cells and 84.6 for MCF10A-KRAS cells, or a ^{˜}4.45-fold increase, which clearly matches the flow cytometry data. Moreover, a number of previously detected proteins were upregulated in the transfected cells. For example EGFR, AXL, FGFR2 and PDGFRA are all upregulated upon KRAS transformation.

### Example 6

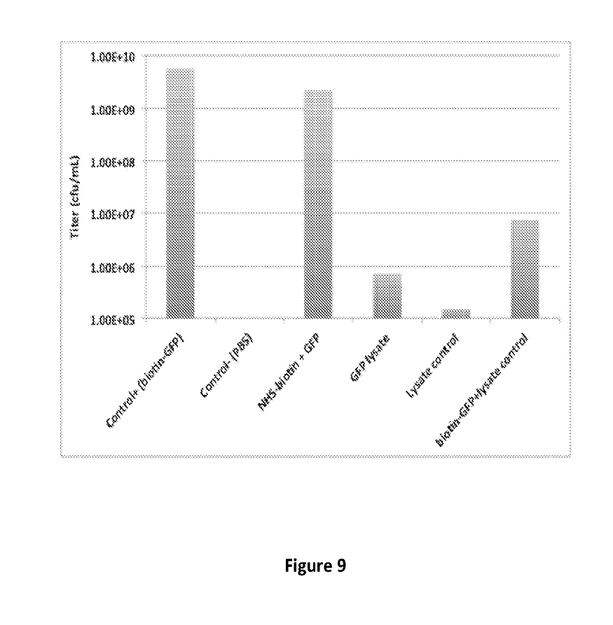

For analysis of intracellular proteins, HEK293T cells expressing intracellular GFP and non-expressing HEK293T cells were collected and lysed using standard procedures. The intracellular proteome was biotinylated using NHS-biotin (N-hydroxysuccinimide biotin), and a buffer exchange was performed to remove the NHS-biotin. The proteins were then immobilized on streptavidin magnetic beads, and the beads were then washed. The Fab-phage library was then applied to the sample according to the techniques and methods described herein and the beads were subsequently washed. The Fab-phages were eluted with acid and the DNA was amplified via propagation of the phage in bacteria. The DNA from the phages were then isolated and amplified and subsequently sequenced. Results are shown in FIG. 9.

### Example 7

A pool containing equal amounts of 192 Fab-Phage clones was created, each with a unique CDRH3, against 58 different membrane protein targets (an average of three to four different Fab-phage clones per target). This PhaNGS library contained mostly receptor tyrosine kinases (RTK), along with a number of other CD proteins and targets of general interest. Each experiment contained negative controls including several non-cognate anti-GFP Fab-phage and intracellular transcription factor Fab-phage including ZNF2, ZNF18, and ZNF343. These phages served as background controls against which the raw values obtained from surface protein Fab-Phage were normalized.

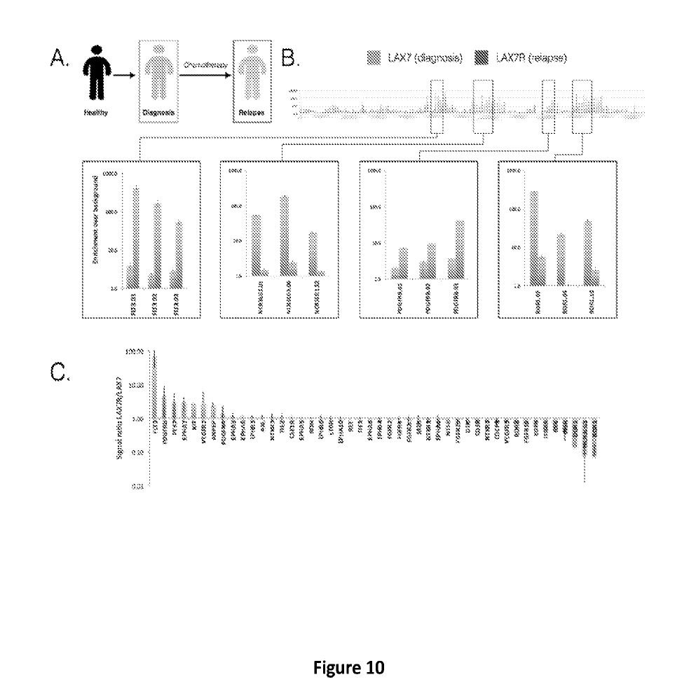

Given the high number of RTKs in the library, B-cells in cancer were profiled. The first set of experiments focused on how B-cells remodel in drug resistance in acute lymphoid leukemia (ALL). B-cell samples were obtained from a patient at diagnosis (LAX7D) and after resistance (LAX7R) to a standard 3-week chemotherapy regimen (vincristine, dexamethasone, L-asparaginase and doxorubicin) (FIG. 10A). Both cell samples contain classical markers of ALL (CD10, CD19 and CD45), but different genetic lesions; the LAX7D has an MLL-AF4 translocation, while LAX7R has a KRAS-G12V mutation that emerged after chemotherapy. Cells originally obtained from the patient were directly engrafted into NOD/SCIDγc−/− mice, and later frozen as stocks. One week before the PhaNGS experiment, samples were thawed and expanded in MEMα growth media to approximately 20 million cells each for quadruplicate analysis.

The PhaNGS profile on these two samples showed a wide range of abundances that vary from the normalized fold-enrichment over background value of 1 to nearly 1000 (FIG. 10B). The standard deviations for technical replicates were small for most of the 192 Fab-Phage clones, suggesting high precision in the measurements. For almost all the targets there were multiple unique Fab-Phage, ranging from three clones in many cases to up to 13 for others. Agreement in fold-change values between antibodies to the same target was observed.

A number of targets changed dramatically between the LAX7D and the LAX7R cells. For example NCR3LG1 and ROR1 were dramatically down-regulated between the diagnosis and relapse samples, while PDGFRB and FLT3 were up-regulated. FLT3 has previously been observed to be over-expressed and/or mutated in ALL and AML, and ROR1 represents a major target of interest in ALL and other leukemias.

In the ALL example, drug resistance developed over the course of a month of treatment and lead ultimately to selection of cells driven by a different genetic abnormality, KRAS-G12V. In addition to genetic changes, studies have also shown that drug treatment of cells can induce resistance via stable metabolic and epigenetic changes.

### Example 8

Myc expression is known to lead to major changes in surface protein expression, thus PhaNGS was performed to detect surface changes between states. For this experiment, an immortalized B-cell line (P493-6) that is engineered to express Myc at high levels, but is repressed by addition of tetracycline (Tet), was chosen to study. This cell line has been used extensively as a model for Burkitt lymphoma, which is a B-cell cancer known to harbor high levels of Myc expression. Using the P493-6 cell line it was possible to toggle Myc expression from ON, to OFF, and BACKON using Tet.

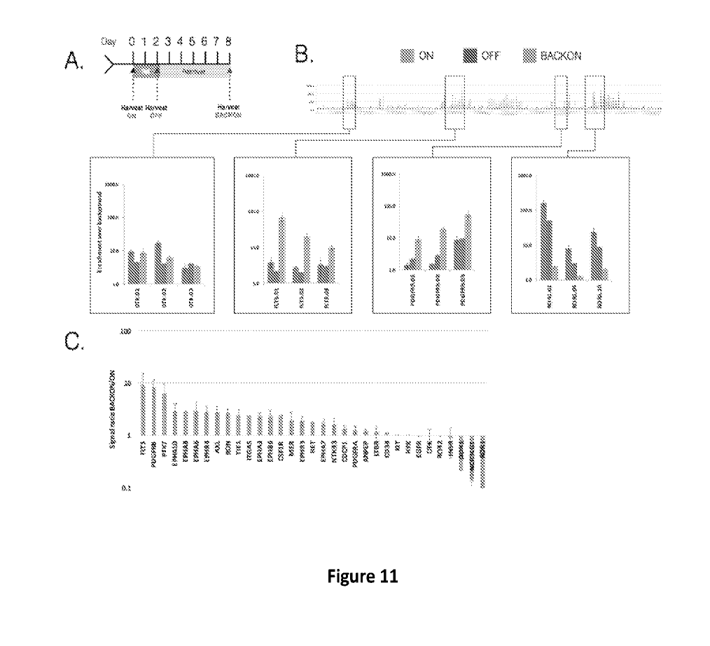

The experiment was carried out over the course of eight days (FIG. 11). Cells initially proliferated rapidly in the absence of Tet, under high Myc expression. Addition of Tet for 2 days repressed Myc expression, and stalled cell growth without apoptosis and lead to subtle changes in morphology. Cells were allowed to recover in the absence of Tet for six days, upon which cell growth and morphology appeared to return to that of the MycON condition. PhaNGS was performed in quadruplicate on the three P493-6 samples representing MycON, MycOFF, and MycBACKON (FIG. 11B). As in the ALL profiles most targets did not change or targets were detected at low levels. About 15% of targets, however, changed dramatically and could be grouped into three categories: (i) a group including DTK and EPHA4 receptor which were high in MycON, expressed lower in MycOFF, and back to the same level in MycBACKON, (ii) a group including FLT3 and PDGFRB that were expressed at modest levels in MycON, went down with MycOFF, and then dramatically up with MycBACKON, and (iii) a group including ROR1, NCR3LG1, FGFR4 and DDR1 that were elevated in MycON, went down with MycOFF, and plummeted further with MycBACKON. These data (FIG. 11C) suggest that the MycBACKON cells did not return to the MycON state even though their growth and morphology appeared to match MycON, but entered a third distinct state. Furthermore, these cellular changes across the culture occured over a matter of days suggesting the changes were unlikely due to genetic selection of resistant cells, but perhaps due to relatively stable epigenetic changes brought on by fluctuating changes in Myc expression.

### Example 9

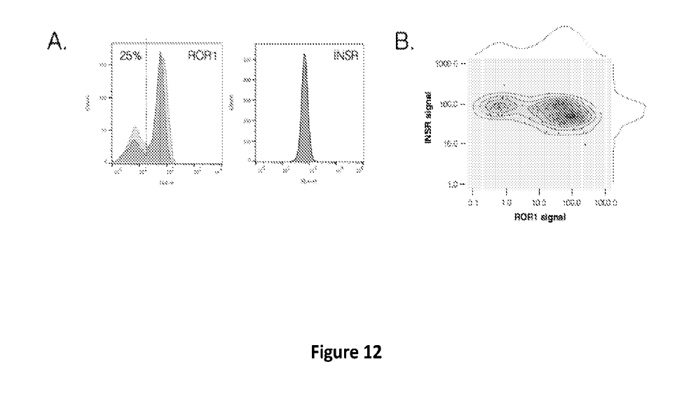

PhaNGS technology was applied to individual cells. P493-6 cells were chosen as the population shows a unimodal distribution of the insulin receptor (INSR) but a bimodal distribution of ROR1 in two replicate flow experiments conducted months apart (FIG. 12A). Five million cells were exposed to Fab-phage to ROR1 and INSR and washed to remove non-binders, similar to the population-level experiments herein. Single cells with bound Fab-phage were rapidly sorted by FACS, and transferred directly into a 96 well plate. The bound Fab-phage were propagated by addition of an E. coli in liquid culture, amplified, and sequenced per the normal method. A robust NGS signal was obtained for each of the two Fab-phage (FIG. 12B), showing close agreement with the flow data. A single, tight distribution of INSR abundance was observed along with ^{˜}25% low-ROR1 and 75% high-ROR1 populations.

### Example 10

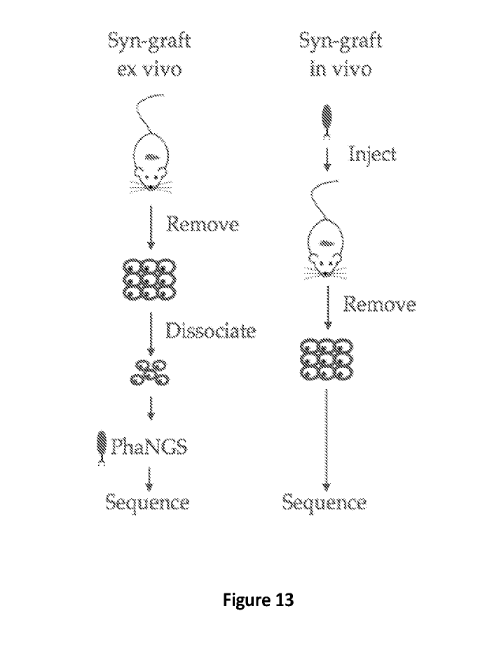

Previously, experiments were performed whereby syn-graft or xenograft experiments involving the engraftment of a human or mouse tumor respectively into a mouse host were allowed to grow, excised, dissociated, subjected to the PhaNGS method to quantify surface protein abundance (FIG. 13, left panel, ex vivo).

The methods of the present invention are used for in vivo identification of proteins (FIG. 13, right panel, in vivo) whereby Fab-phage are injected into the tail vein of a syn-graft (or xenograft) mouse. The Fab-phage are then allowed to circulate, e.g., minutes or hours, before tumor excision and direct phage propagation. In this embodiment, ex vivo washing is not necessary, although ex vivo washing is optional.

The tail vein injection technique was invented decades ago and has been successfully used to select, in most cases, polypeptides which specifically bind to a given organ. The methods of the present invention similarly use an input PhaNGS library to determine, for example, which receptors are highly expressed on a tumor of interest but not on other organs around the mouse's body.

What is claimed is:

1. A method of identifying the presence of one or more proteins in sample, the method comprising
a) applying to a population of proteins in the sample a collection of a multiplicity of Fab-phage particles that contain nucleic acid encoding at least one antibody Fab fragment, wherein each of the antibody Fab fragments of the collection has a predefined specific protein to which it will bind in a specific manner,
b) removing unbound Fab-phage,
c) amplifying the nucleic acid from within the Fab-phages that bound to the proteins, and
d) determining the polynucleotide sequences of the nucleic acids from the Fab-phages that bound to the proteins,
wherein the nucleotide sequences of the nucleic acids from the Fab-phages correlate to the coding sequences of the antibody Fab fragments known to bind in a specific manner to the specific protein.

2. The method of claim 1, wherein the proteins are cell surface proteins and applying the population of proteins in the sample to the Fab-phages comprises mixing intact cells with the Fab-phages.

3. The method of claim 2, wherein the cells are sorted into single cells after applying the Fab-phages to the population of proteins and before the amplifying of the nucleic acid molecules from within the Fab-phages that bound to the cellular proteins.

4. The method of claim 1, wherein the proteins are intracellular proteins and applying the population of proteins in the sample to the Fab-phages comprises lysing a population of cells prior to applying the Fab-phages.

5. The method of claim 1, wherein the proteins are serum protein and the sample comprises serum from a subject.

6. The method of any of the preceding claims, wherein the determining the polynucleotide sequence of the nucleic acid molecules from the Fab-phages comprises indexing the sequences at least one time.

7. The method of any of the preceding claims, wherein the determining the polynucleotide sequence of the nucleic acid molecules from the Fab-phages comprises indexing the sequences at least two times.

8. The method of any of the preceding claims, wherein the applying of a population of proteins to the collection of Fab-phage particles comprises using under-saturated conditions.

9. The method of any of the preceding claims, wherein the applying of a population of proteins to the collection of Fab-phage particles comprises using over-saturated conditions.

10. The method of any of the preceding claims, further comprising quantifying the number of each polynucleotide sequence of the nucleic acid molecules from the Fab-phages that bind to the proteins, such that the specific proteins can be quantified.

11. The method of any of the preceding claims, wherein (a)-(d) are performed at more than one time point such that the presence of specific proteins can be monitored over time.

12. The methods of any of the preceding claims, wherein the nucleic acid molecules from within the Fab-phages comprises at least one H3 region from an antibody CDR.

13. The methods of any of the preceding claims, wherein amplifying the nucleic acid molecules from within the Fab-phages comprises propagating the nucleic acid in a bacteria and isolating the propagated nucleic acid.

14. The method of any of claims 1-12, wherein amplifying the nucleic acid molecules from within the Fab-phages comprise directly amplifying the nucleic acid without first propagating the nucleic acid in a bacteria.

15. The method of any of the preceding claims, wherein the Fab-phage comprises a Fab fragment, an scFv fragment or an affinity reagent.

16. A nucleic acid molecule comprising the polynucleotide sequence of SEQ ID NO:1.

17. A nucleic acid molecule comprising the polynucleotide sequence of SEQ ID NO:2.

18. The nucleic acid of claim 16, wherein the nucleic acid further comprises the polynucleotide sequence of SEQ ID NO:2.

19. The nucleic acid of any of claims 16-18, wherein the nucleic acid further comprises a polynucleotide encoding at least one H3 region from an antibody CDR.

20. A method for identifying one or more target molecules in a mixture comprising (a) contacting a sample with a collection of binding protein DNAs (BPDNAs), (b) removing non-binding BPDNAs, and (c) identifying the BPDNAs bound to the target.

21. The method of claim 20 in which the binding protein or peptide is genetically encoded and displayed from a virus or a cell.

22. The method of claim 20 wherein said BPDNA is derived from phage display

23. The method of claim 21 wherein said BPDNA is derived from yeast display

24. The method of claim 21 wherein said BPDNA is derived from bacterial display

25. The method of claim 21 wherein said BPDNA is derived from mammalian cell display.

26. The method of claim 20 wherein the target is identified by DNA analysis of the bound BPDNA.

27. The method of claim 26 wherein the target is quantified.

28. The method of claim 27 wherein the target is quantified using next generation sequencing.

29. The method of claim 27 wherein the target is quantified using DNA hybridization or any other means of determining DNA sequence.

30. The method of claim 20 wherein said target molecule is a biomolecule.

31. The method of claim 20 wherein said target molecule is a peptide.

32. The method of claim 20 wherein said target molecule is a protein.

33. The method of claim 20 wherein said target molecule is a small molecule.

34. The method of claim 20 wherein said target molecule is a carbohydrate.

35. The method of claim 20 wherein said target molecule is a lipid.

36. The method of claim 32 wherein said protein is a soluble protein.

37. The method of claim 32 wherein said protein is an intracellular protein.

38. The method of claim 32 wherein said protein is an extracellular protein.

39. The method of claim 32 wherein said protein is a plasma-derived protein.

40. The method of claim 20 wherein said sample contains a target in solution.

41. The method of claim 20 wherein said sample contains a target protein attached to an artificial support.

42. The method of claim 20 wherein said sample contains a target protein on a cell surface.

43. The method of claim 20 wherein the binding protein portion of the BPDNA is an antibody or fragment thereof.

44. The method of claim 20 wherein the binding protein portion of the BPDNA is any polypeptide that contains randomized regions within a constant scaffold.

45. The method of claim 20 wherein non-binding BPDNA is removed in a washing step.

46. A method for identifying one or more target molecules in a mixture comprising (a) contacting a sample with a collection of genetically encoded binding polypeptides that are known to bind to specific molecules and are covalently linked to their respective coding gene, (b) removing non-binding polypeptides, and (c) identifying the polypeptide/coding gene complex bound to the target.

47. The method of claim 26 in which the binding polypeptide/coding gene complex is plasmid display or ribosome display.


SEQUENCE LISTING (2 sequences)

SEQ ID NO: 1 - DNA, 72 bp, Artificial Sequence
  Synthetic sequence
  misc_feature (30)..(41): n is a, c, g, or t
aatgatacggcgaccaccgagatctacacnnnnnnnnnnnntgaggacactgccgtctattattgtgctcgc

SEQ ID NO: 2 - DNA, 98 bp, Artificial Sequence
  Synthetic sequence
  misc_feature (63)..(74): n is a, c, g, or t
gactactggggtcaaggaaccctggtcaagatcggaagagcacacgtctgaactccagtcacnnnnnnnnnnnnatctcgtatgccgtcttctgcttg